(19) United States
(12) Patent Application Publication
(10) Pub. No.: US 2023/0272271 A1
(43) Pub. Date: Aug. 31, 2023

(54) Protected Quantum Dots for Therapeutic, Diagnostic, and Other Uses

(71) Applicants: Board of Supervisors of Louisiana State University and Agricultural and Mechanical College, Baton Rouge, LA (US); Quantum Technology Group Inc., Worcester, MA (US)

(72) Inventors: Cristina Sabliov, Baton Rouge, LA (US); Carlos E. Astete, Baton Rouge, LA (US); Siva Sai Ramana Kumar Challa, Newton, MA (US); Eban A. Hanna, Baltimore, MD (US); Jun Wang, Worcester, MA (US)

(73) Assignees: Board of Supervisors of Louisiana State University and Agricultural and Mechanical College, Baton Rouge, LA (US); Quantum Technology Group Inc., Worcester, MA (US)

(21) Appl. No.: 18/017,872

(22) PCT Filed: Jul. 28, 2021
(86) PCT No.: PCT/US21/43372

§ 371 Date: Jan. 25, 2023

Related U.S. Application Data
(60) Provisional application No. 63/060,214, filed on Aug. 3, 2020.

(51) Int. Cl. C09K 11/02; C09K 11/88; A61K 49/00

(52) U.S. Cl. CPC C09K 11/025; C09K 11/883; A61K 49/0067; B82Y 20/00

(57) ABSTRACT

Protected quantum dots are protected from degradation, particularly in aqueous environments, The system comprises quantum dots, hydrophobic core, and hydrophilic shell. The quantum dots are entrapped in and protected by the hydrophobic core. The core polymer is covalently bonded to a hydrophilic shell polymer or protein. Quantum yield is better maintained than for non-encapsulated quantum dots in an aqueous environment. Optionally, ligands are attached to the hydrophilic shell to target delivery of the protected quantum dots, In an alternative embodiment, quantum dots are entrapped in the hydrophilic shell, or in both the shell and the core.

The benefit of the 3 Aug. 2020 filing date of U.S. provisional patent application Ser. No. 63/060,214 is claimed under 35 U.S.C. § 119(e) in the United States, and is claimed under applicable treaties and conventions in all countries.

### TECHNICAL FIELD

This invention pertains to compositions and methods for protected quantum dots for therapeutics, diagnostics, and other uses.

### BACKGROUND ART

Quantum dots (QD) are fluorescent semiconductor nanoparticles with diameters typically from about 2 nm to about 20 nm. Quantum confinement effects arising at these sizes impart unique properties to quantum dots, properties that differ from those of bulk semiconductor materials having the same chemical composition. Quantum dots are often employed as fluorophores. Quantum dots offer advantages over conventional fluorescent molecules, including high photoluminescence, high quantum yield, high photostability, broad absorption spectra, narrow emission spectra, and a large effective Stokes shift. The properties of quantum dots may readily be tuned by modulating their size, shape, or chemical composition.

Changing the size of a quantum dot alters the wavelength of the emitted light due to quantum confinement effects, even when the chemical composition is unchanged and the same excitation wavelength is used. The electron/hole pairs are spatially confined by the dimensions of the QD. Interesting, the QD radius can be shorter than the Bohr radius of the electron-hole pair, or exciton. As the radius of the QD decreases, the band gap energy between the valence band and conduction band increases. Quantum confinement effects are typically more pronounced for semiconductors than for metals, because metals lack the bandgap that is characteristic of semiconductors.

The unique optical properties of quantum dots make them useful for many purposes, including biosensing, bioanalysis, imaging probes, multicolor imaging, QD-based lasers, light emitting devices (LEDs), and photovoltaic cells.

Many QDs have been based on CdSe or CdTe semiconductors. Another material that has been used is ZnSe. Cadmium and other heavy metals are toxic. ZnSe has lower toxicity, making it better suited for biological applications and other applications where toxicity is a concern. In addition, ZnSe has a wide direct band gap (2.7 eV), and a high exciton binding energy, 21 meV, giving it a high stability and high photobleaching resistance for efficient room temperature applications.

Most prior water-soluble quantum dots compositions have been unstable in aqueous environments, especially for long-term storage, due to the relatively rapid oxidation of surface ions, assisted by the water molecules themselves, and the loss of surface ligands. Most prior quantum dot compositions have a hydrophobic coating that is applied during synthesis. When ligand exchange has been used to increase the hydrophobicity of the quantum dot preparations, an unfortunate side effect has been a reduction in the brightness of the quantum dots. The hydrophobicity of prior quantum dot preparations, and their resulting instability in aqueous environments, have limited the use of quantum dots in biomedical applications, such as diagnostics and therapeutics.

There is an unfilled need for protected quantum dots compositions and methods for making protected quantum dot compositions, i.e., compositions that are stable in aqueous environments, making quantum dots better suited for biomedical uses and other uses in aqueous environments.

Tomczak, N., Jańczewski, D., Han, M., & Vancso, G. J. (2009). Designer polymer—quantum dot architectures. Progress in Polymer Sci., 34(5), 393-430. doi:https://doi.org/10.1016/j.progpolymsci.2008.11.004 is a review article describing various approaches that have been tried for making various quantum dot-polymer hybrid materials.

One previous approach has been to synthesize quantum dots directly in an aqueous environment. Although the resulting preparations typically have had relatively narrow ranges of particle size, they often have a spread-out distribution of sizes within that range, leading to wide FWHM (full width at half maximum) of the emission spectrum. See, e.g., Y. K. Lee et al. (2007). Encapsulation of CdSe/ZnS quantum dots in poly(ethylene glycol)-poly(D,L-lactide) micelle for biomedical imaging and detection. Macromolecular Research, 15(4), 330-336. doi:10.1007/BF03218795. Micelles are usually stable only in a liquid phase, and generally dissociate and cannot be stored as stable micelles in a solid state.

Another approach has been to exchange the capping ligands on the QD surface with heterofunctional compounds containing both a hydrophilic end group such as carboxylic acid and a functional group that can form a polar covalent bond with QD surface atoms, such as a thiol. Biomolecules can then be linked to the hydrophilic groups on the QD surface, for example via carbodiimide chemistry. See, e.g., C. Lee et al. (2019). Feasibility of a point-of-care test based on quantum dots with a mobile phone reader for detection of antibody responses. PLOS Neglected Tropical Diseases, 13(10), e0007746. doi:10.1371/journal.pntd 0.0007746.

Ligand exchange has been used to try to protect quantum dots in aqueous environments. However, ligand exchange reactions can negatively affect surface passivation of the QDs, reducing the quantum yield. The reduced quantum yield may result from the formation of surface traps, which provide pathways for nonradiative exciton recombination. Surface traps can also be produced by external stimuli such as heat, oxidation, and moisture. See, e.g., Giansante, C., & Infante, I. (2017). Surface Traps in Colloidal Quantum Dots: A Combined Experimental and Theoretical Perspective. The Journal of Physical Chemistry Letters, 8(20), 5209-5215. doi:10.1021/acs.jpclett.7b02193.

Yoon, C., Yang, K. P., Kim, J., Shin, K., & Lee, K. (2020). Fabrication of highly transparent and luminescent quantum dot/polymer nanocomposite for light emitting diode using amphiphilic polymer-modified quantum dots. Chemical Engineering Journal, 382, 122792. doi:https://doi.org/10.1016/j.cej.2019.122792 reported CdSe@ZnS/ZnS core-shell quantum dots encapsulated with poly(styrene-co-maleic anhydride) (PSMA). The PSMA-QD composites were used as cross-linkers with aminopropyl-terminated polydimethylsulfoxane (PDMS) resin to produce nanocomposite films with QDs, films that might be used for light-emitting diodes.

Ko, J., Jeong, B. G., Chang, J. H., Joung, J. F., Yoon, S.-Y., Lee, D. C., . . . Bang, J. (2020). Chemically resistant and thermally stable quantum dots prepared by shell encapsulation with cross-linkable block copolymer ligands. NPG Asia Materials, 12(1), 19. doi:10.1038/s41427-020-0200-4 discloses CdSe/ZnCdS quantum dots protected in a double shell composite, with a poly(glycidyl methacrylate) (PGMA) inner shell, and a poly(methylmethacrylate) outer shell. The PMMA outer shell imparted miscibility in a PMMA optical film, and the PGMA inner shell passivated surface defects on the QDs to inhibit surface oxidation. The PMMA and the PGMA are both hydrophobic polymers. The PMMA outer shell presumably rendered the composition hydrophobic.

Synthetic polymers have also been used to coat quantum dots. The polymer can be chosen to be transparent at the wavelength(s) of interest, thus minimizing the effect of the polymer on the QD's optical properties. See, e.g., Kumari, A., & Singh, R. R. (2017). Encapsulation of highly confined CdSe quantum dots for defect free luminescence and improved stability. Physica E: Low-dimensional Systems and Nanostructures, 89, 77-85. doi:https://doi.org/10.1016/j.physe.2017.01.031.

Polymeric nanoparticles (PNPs), some based on biopolymers, have been used for various applications, including controlled release of an active ingredient (AI), and protecting an AI from environmental degradation. Depending on the details of their composition, PNPs can have low toxicity, high biodegradability, high biocompatibility, and low cost.

WO/2020/076886 discloses amphiphilic biopolymers synthesized by grafting lignin onto poly(lactic-co-glycolic) acid (PLGA) to form graft polymers, which can then be further assembled into polymeric nanoparticles without a requirement for surfactants. The nanoparticles have a typical diameter of 75 nm. The nanoparticles could be used, for example, for drug delivery.

K. Sill, Quantum dot-polymer nanocomposites: new materials for dispersion, encapsulation, and electronic applications (PhD Dissertation, Univ. Mass. Amherst, 2006) discloses the preparation of CdSe-polymer composites, in which the quantum dots were said to be evenly dispersed throughout the polymer, and in which the polymer component was optionally cross-linked to improve stability. See also Astete, C. E., De Mel, J. U., Gupta, S., Noh, Y., Bleuel, M., Schneider, G. J., & Sabliov, C. M. (2020). Lignin-Graft-Poly(lactic-co-glycolic) Acid Biopolymers for Polymeric Nanoparticle Synthesis. ACS Omega, 5(17), 9892-9902. doi:10.1021/acsomega.0c00168.

Lignin is a hydrophilic, branched, polyphenolic polyether. Lignin is an abundant by-product from the pulp and paper industry. After cellulose, lignin is the second most abundant natural renewable biopolymer. Lignin's properties are useful for many applications, including resistance to decay, resistance to biological attacks, resistance to degradation from ultraviolet or visible light, high stiffness, and resistance to oxidation.

Albumins are a class of globular transport proteins. Serum albumin, produced in the liver, constitutes about half of serum proteins in healthy individuals. Its functions include maintenance of oncotic pressure, and transportation of endogenous and exogenous ligands. Albumin has been incorporated into nanoparticles used for drug delivery vehicles. See Hosseinifar, N., Goodarzi, N., Sharif, A. A. M., Amini, M., Esfandyari-Manesh, M., & Dinarvand, R. (2020). Preparation and Characterization of Albumin Nanoparticles of Paclitaxel-Triphenylphosphonium Conjugates: New Approach to Subcellular Targeting. Drug Res (Stuttg), 70(2-03), 71-79. doi:10.1055/a-1016-6889.

Singh, S., Kaur, R., Chahal, J., Devi, P., Jain, D. V. S., & Singla, M. L. (2013). Conjugation of nano and quantum materials with bovine serum albumin (BSA) to study their biological potential. J. Luminescence, 141, 53-59. doi:https://doi.org/10.1016/j.jlumin.2013.02.042 reported the adsorption of CdS QDs to bovine serum albumin. Fluorescence emission was enhanced when the QDs were excited at 300 nm. This effect was attributed to either the stabilizing effect of the protein on the QD, or energy transfer from tryptophan residues in the albumin to the quantum dots.

Kumari, A., & Singh, R. R. (2017). Encapsulation of highly confined CdSe quantum dots for defect free luminescence and improved stability. Physica E: Low-dimensional Systems and Nanostructures, 89, 77-85. doi:https://doi.org/10.1016/j.physe.2017.01.031 reported that encapsulating CdSe quantum dots in poly-ethylene glycol (PEG) could reduce trap states that would otherwise adversely affect the emission spectra.

Poly(lactic-co-glycolic) acid (PLGA) is a copolymer produced from two natural products: lactic acid and glycolic acid. PLGA has been used for drug delivery applications. PLGA is biodegradable; it hydrolyzes in vivo back into the components, lactic acid and glycolic acid. The rate of PLGA's degradation can be controlled by varying the ratio of lactic acid to glycolic acid; a higher proportion of lactic acid results in slower degradation. The release of active ingredients from a PLGA matrix can also be controlled by selecting the lactic acid:glycolic acid ratio, the molecular weight of the PLGA, or both.

Ranzoni, A., den Hamer, A., Karoli, T., Buechler, J., and Cooper, M. A. (2015). Improved Immunoassay Sensitivity in Serum as a Result of Polymer-Entrapped Quantum Dots: ‘Papaya Particles’. Anal. Chem., 87(12), 6150-6157. doi:10.1021/acs.analchem.5b00762 reported a 40% reduction in fluorescence intensity for CdSe/ZnS quantum dots entrapped in polystyrene (PS) as compared to QDs in free solution.

Lin, Y., Zhang, L., Yao, W., Qian, H., Ding, D., Wu, W., & Jiang, X. (2011). Water-Soluble Chitosan-Quantum Dot Hybrid Nanospheres toward Bioimaging and Biolabeling. ACS Applied Materials & Interfaces, 3(4), 995-1002. doi:10.1021/am100982p reported the preparation of hybrid nanospheres in which ligand exchange was used to modify CdSe/ZnS QDs and entrap them in chitosan. The resulting quantum yield was about 39% of the original value.

Many, although not all, prior approaches to entrapping quantum dots in a polymer matrix have resulted in a reduced quantum yield. While encapsulation can protect quantum dots from oxidation and other types of chemical degradation, the entrapping polymer can itself reduce quantum yields via non-radiative pathways such as electron transfer to adjoining atoms or chemical groups. Ligand exchange also substantially reduces quantum yield. Also, higher QD concentrations can promote irreversible agglomeration of polymeric particles, resulting in physical instability and poor optical properties. Even with these limitations, it has been observed in prior approaches to polymeric encapsulation that the QDs have experienced minimal or no shifts in emission wavelengths, and minimal or no broadening of the FWHM in the emission spectra. However, shifts in the absorption peaks have typically been induced by the encapsulating polymer.

There remains an unfilled need for improved compositions and methods to protect quantum dots, particularly in aqueous environments, without substantially reducing the quantum yield.

### DISCLOSURE OF THE INVENTION

We have discovered compositions and methods for protecting (or passivating) quantum dots, protecting the quantum dots from oxidation and other types of chemical degradation, particularly while permitting the quantum dots to disperse in aqueous environments, and without substantial reductions in quantum yield. The system comprises at least the following three components: quantum dots, hydrophobic polymer core, and hydrophilic polymer or protein shell. The quantum dots are entrapped in a hydrophobic polymer core. The core polymer is covalently bonded to a hydrophilic shell polymer or protein. The quantum dots are protected by the hydrophobic core. The hydrophilic shell is the component principally exposed to the environment. The compositions are typically stable in an aqueous environment. So long as the chemical nature of the polymer(s) themselves does not contribute significantly to fluorescence quenching, the composite structure itself preserves quantum yield, with little or no reduction in quantum yield as compared to that of the free quantum dots. Optionally, ligands or other moieties may be covalently attached to the hydrophilic shell, to target delivery of the protected quantum dots. The luminescence intensity may be varied by varying the ratio of the hydrophilic polymer to the hydrophobic polymer used.

In an alternative embodiment, the quantum dots are physically entrapped in the hydrophilic polymer or protein that comprises the shell, so that the quantum dots are embedded in the hydrophilic shell rather than the hydrophobic core. For example, electrostatic interactions between positively-charged QDs could entrap them within a negatively-charged lignin shell. In another alternative embodiment, two species of quantum dots are entrapped, one in the core, and the other in the shell, leading to unique properties. QDs in the core are better shielded from environmental conditions, but may have somewhat lower quantum yield. QDs in the shell will have a quantum yield that is little changed, but they will likewise also be more exposed to the environmental conditions. However, there can be advantages to entrapping QDs in the shell. For example, a composition with different QDs in the shell and in the core could be used for multifunctional sensing applications. Two types of sensors, one hydrophobic and one hydrophilic, can analyze different environmental factors. For example, a reduction in the brightness of QDs in the outer shell could be an indicator of specific environmental conditions. Also, there is a potential for interactions such as fluorescence resonance energy transfer (FRET) between different QDs in the shell and in the core.

If biopolymers or biocompatible polymers are used in the novel compositions, for example PLGA, albumin, and lignin, then the protected quantum dots may be used in biomedical systems. The quantum dots used in this invention may be any of those known in the art, and preferably are of low toxicity. Particularly when used in biomedical systems, it is preferred to use quantum dots that themselves are nontoxic, such as those formed from ZnSe. Other nontoxic quantum dot materials include, for example, In/P, InP/ZnS, CuInS/ZnS, Si, Ge, and C.

Exemplary methods that may be used to synthesize the quantum dots are described in U.S. Pat. Nos. 8,859,000; 7,608,237; G. Karanikolos et al., “Synthesis and Size Control of Luminescent ZnSe Nanocrystals . . . .” Langmuir, vol. 20, pp. 550-553 (2004); G. Karanikolos et al., “Templated Synthesis of ZnSe nanostructures . . . .” Nanotechnology, vol. 16, pp. 2372-2380 (2005); G. Karanikolos et al., “Water-based synthesis of ZnSe nanostructures . . . .” Nanotechnology, vol. 17, pp. 3121-3128 (2006).

Lignin is a material that may be used in the nanoparticle shell. Although lignin itself may absorb some photons and thereby reduce the quantum yield, the quantum yield is still expected to be significantly higher than that seen with ligand exchange. Lignin will help avoid surface defects that might otherwise reduce quantum yield, and will help protect QD surfaces from oxidation and other degradative reactions, both in vitro and in vivo.

Albumin, on the other hand, is expected to result in higher fluorescence emission, but to be more sensitive to temperature and pH than lignin. Albumin can substantially but reversibly change its conformation in response to changes in pH, with conformational transitions occurring around pH 2.7, 4.3, 8, and 10. This effect can either be a problem or an advantage, depending on how it is used. For example, “smart” nanoparticles can release QDs at certain pH values, while protecting the entrapped QDs from degradation prior to release.

By using a hydrophobic core to entrap QDs, quantum yield can be close to that seen with unmodified, as-synthesized free QDs, while the hydrophilic shell polymer protects the quantum dots in an aqueous environment.

Regardless of the specific polymer(s) used in specific embodiments of the invention, using polymeric nanoparticles rather than ligand exchange to protect quantum dots greatly reduces the incidence of surface traps, and enhances optical properties, particularly the quantum yield. Optionally, functional groups may be incorporated into the polymers to, for example, assist targeting or bio-conjugation.

Sodium lignosulfonate (SLGN) was initially chosen over other forms of lignin such as alkaline lignin (ALN), because SLGN-PLGA has a thinner shell, with an overall nanoparticle size generally above 100 nm. See Astete et al. (2020). With a thinner shell, absorption effects on the system's fluorescence properties can be reduced. In applications where smaller particle sizes may be preferred, however, alkaline lignin (ALN) may be used instead of sodium lignosulfonate (SLGN).

Albumin should result in highly biocompatible particles for in vivo uses in humans or other animals. As an alternative, casein is a low-cost, common food protein that can be used in lieu of albumin. As with albumin, casein is negatively charged at physiological pH, owing to the high fraction of glutamic acid. The zeta potential will be experimentally measured to confirm the charge.

Hydrophobic manganese-doped zinc selenide quantum dots can be synthesized with various method known in the art. Other ZnSe and doped ZnSe quantum dots may be used. Dopants that may be used include, for example, Ag, Cu, or Mn, to produce ZnSe:Mn, ZnSe:Cu, or ZnSe:Ag quantum dots. In addition to quantum dots made from II-VI semiconductors, III-V semiconductors may also be used, for example InP, GaAs, or ternary compositions such as CuInSe2, AgInSe₂, CuInS₂ or AgInS₂.

### BRIEF DESCRIPTION OF THE DRAWING

The drawing depicts schematically an embodiment of the invention.

### MODES FOR PRACTICING THE INVENTION

In certain embodiments, ZnSe quantum dots are protected by entrapment in biodegradable, core-shell nanoparticles such as those comprising core-shell sodium lignosulfonate-poly(lactic-co-glycolic acid) (SLN-PLGA) particles; core-shell albumin-PLGA (ALB-PLGA) particles; or both. The protected quantum dots will be used in applications such as biosensing, therapeutics, microfluidics, diagnostics, and bioimaging.

The drawing depicts schematically an embodiment of the invention. Quantum dots 105 are entrapped in a hydrophobic core polymer 102, which in turn is covalently bonded to hydrophilic shell polymer 104. When the quantum dots fluoresce, they are stimulated by excitation wavelength 101, and they give off photons with emission wavelength 103.

Through routine varying of the following parameters, for a particular application one of skill in the art can readily optimize the effect on optical properties of varying such parameters as: nanoparticle size, shell thickness, colloidal stability, and the hydrophobic/hydrophilic ratio of the graft polymers in the nanoparticles. Also the quantum dot concentration for optimal quantum yield may be determined. The rate of QD leaching into aqueous solution as a function of time will be measured to determine the rate of physical degradation of a particular system. Also, to confirm that quantum dot fluorescence in the novel system is sufficiently resistant to oxidative conditions, the system will be assessed in the presence of differing concentrations of hydrogen peroxide to determine how much, if at all, photoluminescence is affected by an oxidative environment.

Sodium lignosulfonate (SLGN) or bovine serum albumin (ALB) was chosen for the shell in initial embodiments, due to their biocompatibility and hydrophilicity. PLGA was chosen for the core in initial embodiments, due to its biocompatibility and hydrophobicity. SLGN-PLGA and ALB-PLGA each self-assemble into nanoparticles without the need for any extrinsic surfactant. The hydrophobicity of PLGA allows it to entrap hydrophobic quantum dots in the nanoparticle core. Both SLGN-PLGA and ALB-PLGA systems can produce NPs with diameters 100-200 nm. Nanoparticles formed with SLGN-PLGA have a thinner shell than those formed with alkaline lignin. The ratio of albumin or lignin to PLGA can be adjusted through routine experimentation to optimize the hydrophobic-hydrophilic ratio, the shell thickness, and the nanoparticle size, and thereby to optimize the quantum yield.

Point-of-care diagnostics: Point-of-care diagnostics will benefit from the advantages offered by the protected quantum dots. Quantum dots exhibit superior optical properties as compared with conventional organic fluorophores, such as higher brightness, narrow and symmetric emission spectrum, broad Stokes shift, and strong resistance to photobleaching. The protected quantum dots are more resistant to surface oxidization, which further enhances their stability.

Conventional fluorophores are sensitive to pH, temperature, light, and ionic strength. The novel protected quantum dots typically have higher stability over a wider range of pH, temperature, and ionic strengths (depending of course on the specific composition(s) used). Also, the higher surface area of the nanoparticle shell, as compared to the surface area of the quantum dots themselves, optionally permits relatively easy conjugation to biomolecules, especially to larger proteins. For example, biofunctionalized nanoparticles with entrapped QDs can be used for in vitro and in vivo biomedical imaging and diagnostics.

In Vivo Diagnostics: Biodegradability, biocompatibility, low toxicity, and high sensitivity are desirable for uses with in vivo diagnostics. Organic polymers such as poly(lactic-co-glycolic acid), poly(butyl cyanoacrylate), poly(alkyl cyanoacrylate), poly(ethyl cyanoacrylate), and others may be used in the novel compositions, because they are biodegradable, biocompatible, and compatible with a broad range of products for in vivo diagnostics. These new materials for in vivo diagnostics can have a biodegradable and biocompatible surface, high brightness, resistance to photodegradation, multiplexing with single laser excitation, high sensitivity, high specificity, and high detection efficiency.

Our initial attempts to prepare protected quantum dots, along the lines generally described, were unsuccessful. Our initial attempts either failed to entrap the quantum dots in the nanoparticles, or the initially-entrapped quantum dots were expelled from the nanoparticles during purification, before the compositions might be used. We reconsidered our approach, refined our procedures, and in subsequent efforts we were successful in producing protected, purified quantum dot preparations.

The polymeric core-shell nanoparticles were prepared as otherwise generally provided in WO/2020/076886—except for the modifications described herein, which we employed to successfully entrap the quantum dots in the nanoparticles, and then to purify the resulting preparations or alternatively to make a purification step unnecessary.

In certain embodiments polymers that are used in the hydrophobic core of the nanoparticles may include one or more of poly(lactic-co-glycolic) acid (PLGA), polystyrene, polyhydroxyalkanoates, polylactic acid, poly glycolic acid, poly(methyl methacrylate), ammonio methacrylate, polystyrene, poly(styrene-co-maleic anhydride), polyethylene, and poly(propylene oxide). For in vivo applications, the polymers of the core are preferably nontoxic to mammals.

In certain embodiments polymers and proteins that are used in the hydrophilic shell of the nanoparticles may include one or more of zein, soy protein, poly(ethylene glycol) (PEG), poly(vinyl alcohol) (PVA), poly(glutamic acid), sodium lignosulfonate (SLGN), bovine serum albumin (ALB), alkaline lignin, polyacrylamide, polyethyleneimine, collagen, substituted or unsubstituted cellulose, substituted or unsubstituted starch, and polynucleotides. The polymers or proteins of the shell are preferably nontoxic to mammals.

The quantum dots comprise a semiconductor. In certain embodiments, the quantum dots luminesce/fluoresce with excitation in the ultraviolet spectrum and emission in the ultraviolet or visible spectrum. For some applications, the quantum dots are preferably nontoxic to mammals; while in other applications toxicity may be less of a concern. In certain embodiments, the quantum dots have a mean diameter from 2 nm to 20 nm. In certain embodiments, the quantum dots comprise ZnSe or doped ZnSe quantum dots, for example ZnSe:Mn, ZnSe:Cu, or ZnSe:Ag. In certain embodiments, the quantum dots comprise graphene QDs, carbon QDs, far infrared QDs, zinc-based QDs, or other metal-based QDs; or III-V quantum dots such as InP; or ternary composition quantum dots such as CuInSe₂, CuInS₂, AgInSe₂, or AgInS₂.

In certain embodiments the nanoparticles have a mean diameter from 100 nm to 250 nm. In certain embodiments the nanoparticles have a mean diameter from 70 to 500 nm.

In certain embodiments the quantum dots are predominantly located inside the nanoparticles; wherein the quantum dots associate primarily with the inner, hydrophobic core of the nanoparticles if the surface of the quantum dots is hydrophobic and not electrostatically charged; and wherein the quantum dots associate primarily with the outer, hydrophilic shell of the nanoparticles if the surface of the quantum dots is hydrophilic or electrostatically charged.

In certain embodiments the quantum dots are protected within the composition; meaning that in an aqueous environment, as compared to free quantum dots that are otherwise chemically identical but that lack the graft copolymer and the nanoparticles, the degradation rate of the quantum dots within said composition is slower by a factor of at least 1.25, or at least 1.5, or at least 2, or at least 3, or at least 5, or at least 8, or at least 10.

### Example 1: Synthesis of PLGA-Biotin Conjugate

PLGA was functionalized by conjugating it to polyethylene glycol-biotin (MW 5,000) in a two-step acylation. In the first step, PLGA carboxylic end groups were activated with oxalyl chloride (4 hours at room temperature). In the second step, the activated PLGA reacted with PEG-biotin (24 hours at room temperature) to form PLGA-biotin conjugates (PLGA-Bio). The products were washed with ethyl ether and ethanol. The white precipitate was dried under high vacuum for 24 hours, and then stored at −20° C. until used.

### Example 2: Unsuccessful Attempt to Prepare and Purify Protected Quantum Dots with PLGA-Bio

Our first attempt to prepare and purify protected quantum dots was unsuccessful. We attempted to entrap quantum dots in PLGA-Bio nanoparticles using an emulsion-evaporation technique, and to purify the nanoparticles by centrifugation.

Briefly, The PLGA-Bio polymer from Example 1 was dissolved in ethyl acetate (organic phase) for 30 minutes. Next, ZnSe:Mn (ZnSe doped with Mn) quantum dots (QD) were added to the organic phase (10% ratio by weight QD:PLGA-Bio), and mixed at room temperature for another 30 minutes. Then the organic phase was added to an aqueous phase containing 2% by weight poly (vinyl alcohol) (PVA). The two phases were emulsified by homogenization in a microfluidizer M-110P (Microfluidics Corp., Westwood, Mass.) in four passes at 30,000 psi. The solvent was then evaporated in a Rotavapor R-300 (Buchi Inc, Newcastle, Del.) under vacuum at 33° C. for 2 hours. Next, the sample was centrifuged at 29,000 rpm (Beckman Coulter, Indianapolis, Ind.) for 2.5 hours at 4° C. to purify the nanoparticles. The resulting pellet was resuspended in water using a bath sonicator for 20 minutes. Finally, the polymeric nanoparticle suspension was freeze-dried for 2 days (Labconco, Kansas City, Mo.) at −80° C. Trehalose was added as a cryoprotectant at 1:1 mass ratio.

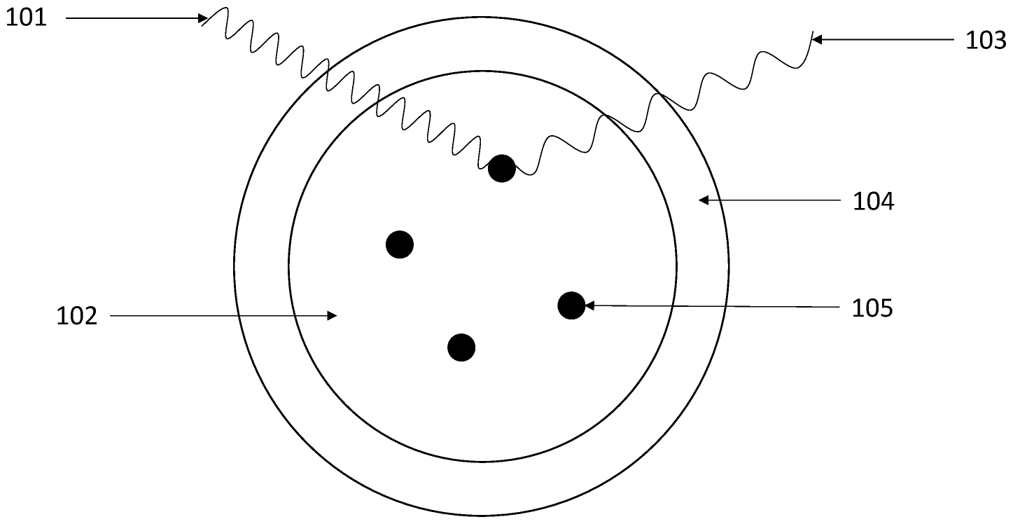

The resulting nanoparticles were examined by transmission electron microscopy. (Photographs are not shown here, but may be viewed as FIG. 1 in priority application 63/060,214). The PLGA-Bio nanoparticles presented a spherical shape, with a mean size of 156±6.4 nm, a polydispersity index of 0.232±0.031, and a zeta potential of −23.4±2.9 mV based on dynamic light scattering (DLS) (See FIG. 2 in priority application 63/060,214).

The TEM micrographs clearly showed both the polymeric nanoparticles as larger gray spheres (˜100 nm), and the quantum dots as smaller black dots (˜5 nm). However, the quantum dots were not entrapped in the polymeric nanoparticles, but were clearly located outside the nanoparticles. Our initial attempt either had failed to entrap the quantum dots, or the quantum dots may have initially been entrapped, but then subsequently migrated from the nanoparticles, perhaps during centrifugation.

### Example 3: Successful Preparation and Purification of Protected Quantum Dots with PLGA-Bio

We hypothesized that the failure in Example 2 had occurred during the purification step. Our hypothesis was that the initial entrapment had likely worked as intended, but that subsequently the quantum dots had migrated from the interior of the nanoparticles during the centrifugation step, due to the higher density of the semiconductor QDs.

To test this hypothesis and to try to avoid the hypothesized centrifugation issues, we next attempted instead to purify the quantum dot/nanoparticle composites by dialysis. This attempt proved to be successful. The quantum dots were successfully entrapped in the nanoparticles, and following dialysis the quantum dots remained in the purified nanoparticles.

The composites were prepared as otherwise described in Example 2, up to but not including the centrifugation step. No centrifugation was performed, and instead the sample was dialyzed after the organic solvent had evaporated.

The PLGA-Bio-Quantum Dot sample was dialyzed for 30 hours (SpectraPor regenerate CE, MW cutoff 300 KD) at room temperature in low resistivity water. The water was changed every 6 hours. After dialysis, the suspension was freeze-dried for 2 days (Labconco, Kansas City, Mo.) at −80° C. Trehalose was added as cryoprotectant at a 1:1 mass ratio.

As seen by Transmission Electron Microscopy, the polymeric nanoparticles had a spherical shape (See FIG. 3 in priority application 63/060,214), with a mean size of 108±1.7 nm, a polydispersity index of 0.228±0.031, and a zeta potential of −25.6±3.1 mV. The TEM micrographs showed that the smaller, denser quantum dots were indeed entrapped inside the polymeric nanoparticles. Confocal microscopy (excitation 350 nm, emission 590 nm) also confirmed entrapment of the quantum dots by fluorescence. The confocal microscopy observations allowed visualization of the location of the quantum dots, and gave a qualitative indication of their fluorescence intensity. With free quantum dots, one would expect to see an agglomeration of fluorescence. With entrapped quantum dots, the fluorescence would be expected to appear more randomly distributed.

### Example 4: Successful Preparation of Protected Quantum Dots Using PLGA-Lignin, without a Separate Purification Step

We synthesized an alkaline lignin-PLGA (ALN-PLGA) co-polymer by acylation in a two-step reaction: first the carboxylic end groups of PLGA were activated, and then lignin was covalently attached. The lignin-PLGA co-polymer can self-assemble into nanoparticles in aqueous solution without the need for extrinsic surfactants. Without extrinsic surfactants, the mixture does not require a separate purification step such as centrifugation or dialysis.

Briefly, 2 g PLGA were dissolved in 30 mL of DCM at room temperature in a three-neck round-bottom flask. A nitrogen flow was then connected to a bubbler bottle with 1 M NaOH to neutralize HCl produced during the reaction. After complete dissolution of PLGA at room temperature, 5 equivalents of oxalyl chloride were added dropwise with a glass syringe. The reaction was performed at room temperature with mild stirring for 4 hr. Following the reaction, the solution was concentrated with a rotavapor Buchi R-300 (Buchi Corporation, New Castle, Del.). Once the solution became viscous during evaporation, 20 mL of DMSO was added and the remaining DCM was evaporated. Afterwards, the second reaction proceeded by adding 500 mg of ALN to 20 mL of DMSO. The PLGA-CI solution was then added to the ALN solution. The reaction was allowed to continue overnight at room temperature under nitrogen flow. The (ALN-graft-PLGA) polymer was then precipitated by adding 150-200 mL of ethyl ether, and washed three times with ethyl ether. The precipitated polymer was then suspended in 20 mL of DCM, and the organic phase was washed with water to remove unreacted lignin to obtain a clear supernatant. Finally, DCM was evaporated with a rotavapor Buchi R-300, and the polymer was dried under high vacuum for 3 days at 30° C. The ALN-PLGA copolymer was stored at 2-4° C. until used for nanoparticle synthesis.

Nanoparticles were prepared from the synthesized LGN-PLGA polymer by emulsion evaporation. Briefly, the LGN-PLGA polymer was dissolved in ethyl acetate (organic phase) and QDs were added with 20 min of mixing. The organic phase was added to 50 mL deionized water with 5 mL ethyl acetate (aqueous phase). An emulsion was then prepared with a Microfluidics M-110P (Microfluidics Corp., Westwood, Mass.). Next, the solvent was evaporated with Rotavapor Buchi R-300 (Buchi Inc., New Castle, Del.). The sample was then freeze-dried for 2 days, with trehalose added as cryoprotectant at 1:1 mass ratio. The freeze-dried powder was later resuspended in water and characterized.

TEM micrographs showed polymeric nanoparticles having a spherical shape, with a white spherical core of hydrophobic PLGA surrounded by a grey shell of the more hydrophilic lignin. (Photographs are not reproduced here, but they may be viewed as FIG. 6 in priority application 63/060,214.) The QDs were efficiently entrapped in the core, seen as the grey speckles in the white PLGA core. No dark spots were detected outside the particles, as we had seen for the centrifuged PLGA-Bio-QD preparation. The nanoparticles had a mean size of 102.7±5.3 nm, a PDI of 0.189±0.021 and a zeta potential of −68.3±4.3 mV, based on DLS measurements. Fluorescence of the entrapped QDs was confirmed by confocal microscopy (excitation 350 nm, emission 590 nm).

### Example 5. SLN-PLGA Copolymer Synthesis

The SLN-PLGA copolymer is synthesized via a method adapted from Astete et al. (2020). Briefly, 2 g of PLGA are dissolved in 30 mL of DCM at room temperature in a three-neck round-bottom flask. A nitrogen flow is then connected to a bubbler bottle with 1 M NaOH to neutralize HCl evolved from the reaction. After complete dissolution of PLGA at room temperature, 5 equivalents of oxalyl chloride are added dropwise with a glass syringe. The reaction is held at room temperature with mild stirring for 4 hours. Following the reaction, the solution is concentrated with a Buchi R-300 rotavapor (Buchi Corporation, New Castle, Del.). Once the solution becomes viscous during evaporation, 20 mL DMSO is added, and the remaining DCM is evaporated. Then the second reaction proceeds by adding 500 mg of SLN to 20 mL of DMSO. The PLGA-Cl solution is then added to the SLN solution. The reaction is held overnight at room temperature under nitrogen flow. The (SLN-graft-PLGA) polymer is then precipitated by adding 150-200 mL ethyl ether, and the precipitate is washed three times with ethyl ether. The precipitated polymer is suspended in 20 mL DCM, and the organic phase is washed with water to remove any unreacted lignin and to obtain a clear supernatant. Finally, DCM is evaporated with a Buchi R-300 rotavapor, and the polymer is dried under high vacuum for 3 days at 30° C. The SLN-PLGA copolymer is stored at 2-4° C. until used.

### Example 6. ALB-PLGA Copolymer Synthesis

The synthesis of ALB-PLGA generally follows the SLN-PLGA synthesis as described in Example 5 above, with some modifications. The first reaction (activation of PLGA) is essentially the same. The second reaction begins by adding 500 mg albumin to 20 mL of DMSO. After dissolution, the PLGA-Cl solution is pipetted into the ALB solution. The reaction is performed for 24 hours at room temperature under nitrogen flow. The ALB-graft-PLGA polymer is then precipitated with the addition of 150-200 mL of ethyl ether, and is washed three times with ethyl ether. The precipitated polymer is suspended in 20 mL DCM, and the organic phase is washed with water to remove unreacted albumin and obtain a clear supernatant. Finally, DCM is evaporated with a rotavapor Buchi R-300, and the polymer is dried under high vacuum for 3 days at 30° C. The ALB-graft-PLGA polymer is stored at 2-4° C. until used.

### Examples 7 and 8. SLN-PLGA Nanoparticle Synthesis, and ALB-PLGA Nanoparticle Synthesis

Biopolymer nanoparticles are synthesized by the emulsion evaporation technique of Astete et al. (2020). No extrinsic surfactants are added, and thus no purification step is required. Briefly, 150-500 mg of SLN-PLGA or ALB-PLGA is dissolved in 5 mL ethyl acetate at room temperature with strong stirring. Next, the organic phase is added to the aqueous phase (50 mL of deionized water (DI) water with 5 mL of ethyl acetate). After 10 min of mixing, the suspension is homogenized with a microfluidizer (Microfluidics Corp., Westwood, Mass.) at 30,000 psi, four times at 4° C. Afterwards, the organic solvent is evaporated in a rotavapor R-300 (Buchi Corporation, New Castle, Del.) at 32° C. under vacuum for at least 45 min. Finally, trehalose is added (1:1 mass ratio) as a cryoprotectant, and the samples are placed in a freeze-drier FreeZone 2.5 (Labconco Corporation, Kansas City, Mo.) for 2 days at −80° C. to remove water. The biopolymer nanoparticle samples are stored at 4° C. until they are characterized or used.

### Examples 9-14. Protection of QDs by Entrapment in SLN-PLGA or ALB-PLGA Nanoparticles

SLN-PLGA and ALB-PLGA polymers are synthesized, as otherwise described above, at three different SLN:PLGA or ALB:PLGA mass ratios—namely, 2:1, 1:1, and 1:2 (w/w).

NP(QD) conjugates are then prepared. A preferred NP(QD) composite is then selected as the sample with the highest quantum yield, so long as it also satisfies the following two criteria: (1) a relatively homogeneous distribution of QDs among the NPs; and (2) NP(QD) composite diameter between 100 and 250 nm.

Different QD amounts (equal to 10%, 30%, or 50% of the graft polymer, by mass) are entrapped in polymeric nanoparticles: 150-500 mg of the SLN-PLGA or ALB-PLGA polymer is dissolved with QDs in 5 mL of ethyl acetate at room temperature. This organic phase is then added to 50 mL of DI water (aqueous phase) and stirred for 10 minutes. The suspension is homogenized with a microfluidizer (Microfluidics Corp., Westwood, Mass.) at 30,000 psi, four times at 4° C. The organic solvent is then evaporated from the suspension at 33° C. under vacuum with a Rotavapor R-300 (Buchi Inc, New Castle, Del.) for 2 hours. Trehalose is then added to the particle suspension at a 1:1 mass ratio before freeze-drying for 2 days at −80° C. The resulting powder is stored at 4° C. until it is characterized or used.

To determine entrapment efficiency, the particles are washed three times with hexane and a membrane filter (Novamem Membrane Filters, PEEK20, 0.02 Micron, MF, 47 mm, 25/Pk). The supernatant is collected, and the fluorescence intensity is measured. A standard curve is generated to correlate the observed fluorescence to the QD concentration.

### Examples 15-20. Optical and Physical Characterization of SLN-PLGA and ALB-PLGA NP(QD) Conjugates

A preferred NP(QD) system is one whose particles have a suitable size (e.g., 100-250 nm diameter), and that retains the optical properties of the as-synthesized quantum dots, without change or with only minor changes.

Nanoparticles 10-100 nm can enter lymphatic capillaries and there undergo clearance. Thus the particles should preferably have a diameter 100 nm or larger. Particles 250 nm-1 μm can be endocytosed by macrophages, and then be removed by the reticuloendothelial system. Thus the particles preferably have a diameter 250 nm or smaller. Taking both factors together, a preferred size range for the conjugates is 100-250 nm diameter.

The composition of the polymeric nanoparticle can potentially affect the optical properties of the entrapped quantum dots. The effect of particle composition and quantum dot concentration on the size, zeta potential, polydispersity index (PDI), conjugate structure, quantum yield, and absorption/emission spectra are measured to ensure that these properties all remain within acceptable bounds. The properties of the conjugates can be characterized, for example, using techniques otherwise known in the art for dynamic light scattering (DLS), cryogenic transmission electron microscopy (Cryo-TEM), X-ray powder diffraction (XRD), UV-Vis spectrophotometry, fluorescence spectrophotometry, and fluorescence microscopy. DLS is used to measure size, polydispersity index, and zeta potential. The morphology of the nanoparticles and their core-shell structure are analyzed by TEM. The structure of the NPs is further characterized by XRD. Absorption and fluorescence spectra are measured by UV-Vis spectrophotometry and fluorescence spectrophotometry, respectively. Images of the conjugates' photoluminescence intensity are taken with fluorescence microscopy.

To determine quantum yield, UV-Vis absorbance measurements are made at different concentrations of the QD-NP suspension, and of a standard for comparison (anthracene or 2-aminopyridine). It is preferred that the absorbance maximum be less than about 0.1 to minimize reabsorption and other non-linear effects. Fluorescence measurements are also taken for each sample. The fluorescence intensity is integrated and plotted against the absorbance for each sample to determine a gradient. The resulting measurements should (approximately) fit the following relationship:

$${Φ}_{X} = {Φ}_{ST} ( \frac{{Grad}_{X}}{{Grad}_{ST}} ) ( \frac{{η}_{X}^{2}}{{η}_{ST}^{2}} )$$

wherein X and ST denote the sample and standard, respectively; Φ is the fluorescence quantum yield; Grad is the gradient from the respective plot (i.e., the slope of the plot of fluorescence versus absorbance), and η is the refractive index of the solvent.

Absorbance and emission spectra will be obtained: for the empty NPs, for the as-synthesized QDs, and for the QD-NP conjugate. A curve of NP concentration versus photoluminescence (PL) will help assess a preferred concentration for the highest PL intensity.

### Examples 21-26. Measuring the Degradation/Stability and QD Leaching of SLN-PLGA(QD) Conjugates and ALB-PLGA(QD) Conjugates Under Different Conditions of pH, Temperature, and Oxidation Potential

The physical stability and the photostability of QDs delivered with the novel conjugates are measured at different pH, temperatures, and oxidation potentials. The stability/degradation properties are important, for example, when used with drug delivery tracking devices or biosensors. The optical properties of free QDs in solution degrade over time due to oxidation and other mechanisms. The NP-entrapped QD conjugates are better protected from degradation, and thus better maintain their photoluminescence properties for a longer time than do free QDs in solution. Degradation of the optical properties of free QDs is believed to occur primarily when the QD surface is directly exposed to solvent. Thus degradation in the novel conjugates is expected primarily once the nanoparticles have degraded to the point where the QDs are no longer effectively entrapped. (For the loss of photoluminescence properties, the diffusion of quantum dots within the polymeric matrix is expected to be less significant than the degradation of the entrapping nanoparticles.) The optical properties of the novel conjugates are observed under various conditions of pH, temperature, and oxidation potential, to assess the rate of their physical degradation and quenching.

A kinetic study determines the physical degradation rate of the conjugates at different pH, for example, at pH 5, 7, and 9, at a constant temperature, for example 25° C. We also observe physical degradation at different temperatures, for example 25° C. and 37° C., at a constant pH, for example pH 7. First 15 mL of a suspension of the conjugate is prepared in a 20 mL vial with magnetic stirring. A 0.2 μL sample is then withdrawn from the vial every hour over the initial 12 hours; and thereafter a 0.2 μL sample is withdrawn every 24 hours over 2 weeks. The method for determining QD encapsulation efficiency for the withdrawn aliquots is essentially as discussed above. Samples are washed three times with hexane using a membrane filter (Novamem Membrane Filters, PEEK20, 0.02 Micron, MF, 47 mm), and the supernatant is collected. Samples are analyzed by fluorescence spectrophotometry to determine the rate of QD leaching from the NP. Using the standard curve previously generated, the concentration of QDs in the supernatant is determined as a function of time. These data are fitted to zero-order, first-order, and second-order kinetic models to determine a best fit, and to make inferences about the apparent degradation mechanism:

$$Zero order \text{:} [ QD ] = {[ QD ]}_{0} - kt$$

$$First order \text{:} ln [ QD ] = {ln [ QD ]}_{0} - kt$$

$$Second order : \frac{1}{[ QD ]} = \frac{1}{{[ QD ]}_{0}} + kt$$

where [QD] denotes the QD concentration at a given time, [QD]₀ is the initial QD concentration, k is the leaching rate constant, and t is the elapsed time.

Hydrogen peroxide quenches fluorescence by oxidation of the quantum dots. Hydrogen peroxide is used to assess the susceptibility of the conjugates to oxidation as compared to the as-synthesized QDs. The conjugates are diluted to 10 nM in DI water in a black, 96-well microtiter plate. (Nalge Nunc, Rochester, N.Y.) H₂O₂ is then added to a final concentration of 0.1-50 μM, and the H₂O₂ is allowed to react with the conjugates for 0, 12, 24, and 48 hours. After the specific time points (i.e. 0, 12, 24, and 48 hours) the concentration of H₂O₂ is increased gradually to 50 μM. The microplate is stored in darkness at room temperature, and it is covered with parafilm between additions of hydrogen peroxide. The plate is read on a fluorescence microplate reader. The temperature is held constant at 25° C., and the oxidation study is repeated at pH 5, 7, and 9. The observed quenching is fitted to the Stern-Volmer equation:

$$\frac{{F}_{0}}{F} = 1 + {K}_{SV} [ Q ]$$

wherein F₀ and F denote the fluorescence intensities in the absence and presence of hydrogen peroxide, respectively; K_{SV} is the Stern-Volmer quenching constant; and [Q] represents the concentration of the quencher, hydrogen peroxide.

### Examples 27-32: Expected Results

Increasing the QD concentration is expected to gradually increase PL intensity up to a maximum, after which the proximity of QDs to one another within the NP matrix will lead to self-quenching reabsorption between neighboring particles. The thickness of the nanoparticle shell also affects photoluminescence; a thinner shell is expected generally to correspond with higher PL values. On the other hand, the properties of albumin may enhance overall fluorescence properties.

### Examples 33-36: Core-Shell Nanoparticles

Our studies of SLN-PLGA and ALB-PLGA nanoparticles (without quantum dots) have confirmed that they successfully form core-shell structures. For example, a 1:2 (w/w) SLN-PLGA system has formed nanoparticles with an average diameter of 102.7±5.3 nm, a polydispersity index (PDI) of 0.189±0.021, and a zeta potential of −68.3±4.3 mV. (Nanoparticles were imaged and measured by TEM and dynamic light scattering.)

As another example, a 1:2 (w/w) ALB-PLGA system formed nanoparticles with an average diameter of 137.9±0.4 nm, a PDI of 0.084±0.007, and a zeta potential of 42.0±0.5.

As another example, a 1:1 (w/w) SLN-PLGA system formed nanoparticles with an average shell thickness of 50.98±8.84 nm, an average diameter of 229.7±2.03 nm, a PDI of 0.164±0.021, and a zeta potential of −43.7±6.03 mV.

### Examples 37-43: Dynamic Light Scattering and Transmission Electron Microscopy Measurements

SLN-PLGA “empty” nanoparticles were prepared at three different ratios of SLN to PLGA (1:2, 1:1, and 2:1). Also prepared were 1:1 SLN-PLGA nanoparticles with entrapped quantum dots. Table 1 summarizes the results of measurements taken with dynamic light scattering and transmission electron microscopy. Both the particle diameter and the zeta potential decreased as the SLN to PLGA ratio changed from 1:2 to 2:1. The shell thickness decreased with increasing ratios of lignin. Addition of QDs changed the measured properties somewhat.

TABLE 1

| | | | | 1:1 |
|---|---|---|---|---|
| | 1:2 | 1:1 | 2:1 | SLN-PLGA |
| | SLN-PLGA | SLN-PLGA | SLN-PLGA | with QDs |
| Particle | 556 ± 15 | 230 ± 2 | 145.6 ± 0.2 | 253.2 ± 0.4 |
| Diameter | | | | |
| (nm) by DLS | | | | |
| Particle | 519 ± 132 | 197 ± 34 | 125 ± 22 | |
| Diameter | | | | |
| (nm) by TEM | | | | |
| PDI by DLS | 0.21 ± 0.02 | 0.16 ± 0.02 | 0.15 ± 0.02 | 0.05 ± 0.02 |
| Zeta | −54.1 ± 1.5 | −45.2 ± 2.4 | −40.8 ± 0.3 | −44.0 ± 0.9 |
| potential | | | | |
| (mV) by DLS | | | | |
| Core | 387 ± 115 | 115 ± 27 | 89 ± 21 | |
| Diameter | | | | |
| (nm) by TEM | | | | |
| Shell | 66 ± 16 | 41 ± 11 | 18 ± 3 | |
| thickness | | | | |
| (nm) by TEM | | | | |

### ADDITIONAL REFERENCES

Baler, K., Martin, 0. A., Carignano, M. A., Ameer, G. A., Vila, J. A., & Szleifer, I. (2014). Electrostatic Unfolding and Interactions of Albumin Driven by pH Changes: A Molecular Dynamics Study. The Journal of Physical Chemistry B, 118(4), 921-930. doi:10.1021/jp409936v
Bao, Z., Jiang, Z.-F., Su, Q., Chiu, H.-D., Yang, H., Chen, S., . . . Liu, R.-S. (2020). ZnSe:Te/ZnSeS/ZnS nanocrystals: an access to cadmium-free pure-blue quantum-dot light-emitting diodes. Nanoscale, 12(21), 11556-11561. doi:10.1039/DONR01019G
Beisl, S., Friedl, A., & Miltner, A. (2017). Lignin from Micro- to Nanosize: Applications. International Journal of Molecular Sciences, 18(11), 2367. Retrieved from https://www.mdpi.com/1422-0067/18/11/2367
Bendtsen, L., Lorenzen, J., Gomes, S., Liaset, B., Holst, J., Ritz, C., . . . Astrup, A. (2014). Effects of hydrolysed casein, intact casein and intact whey protein on energy expenditure and appetite regulation: A randomised, controlled, cross-over study. The British journal of nutrition, 112, 1-11. doi:10.1017/S000711451400213X
Chan, Y.-H., Ye, F., Gallina, M. E., Zhang, X., Jin, Y., Wu, I. C., & Chiu, D. T. (2012). Hybrid Semiconducting Polymer Dot—Quantum Dot with Narrow-Band Emission, Near-Infrared Fluorescence, and High Brightness. Journal of the American Chemical Society, 134(17), 7309-7312. doi:10.1021/ja3022973
Drbohlavova, J., Adam, V., Kizek, R., & Hubalek, J. (2009). Quantum dots—characterization, preparation and usage in biological systems. Int J Mol Sci, 10(2), 656-673. doi:10.3390/ijms10020656
Drobintseva, A. O., Matyushkin, L. B., Aleksandrova, O. A., Drobintsev, P. D., Kvetnoy, I. M., Mazing, D. S., . . . Musikhin, S. F. (2015). Colloidal CdSe and ZnSe/Mn quantum dots: Their cytotoxicity and effects on cell morphology. St. Petersburg Polytechnical University Journal: Physics and Mathematics, 1(3), 272-277. doi:https://doi.org/10.1016/j.spjpm.2015.11.003
Hines, D. A., Becker, M. A., & Kamat, P. V. (2012). Photoinduced Surface Oxidation and Its Effect on the Exciton Dynamics of CdSe Quantum Dots. The Journal of Physical Chemistry C, 116(24), 13452-13457. doi:10.1021/jp303659g
Jang, H.-S., Cho, H.-S., Uhrig, D., & Nieh, M.-P. (2017). Insight into the interactions between pyrene and polystyrene for efficient quenching nitroaromatic explosives. Journal of Materials Chemistry C, 5(47), 12466-12473. doi:10.1039/C7TC04288D
Lomis, N., Westfall, S., Farandel, L., Malhotra, M., Shum-Tim, D., & Prakash, S. (2016). Human Serum Albumin Nanoparticles for Use in Cancer Drug Delivery: Process Optimization and In Vitro Characterization. Nanomaterials (Basel), 6(6). doi:10.3390/nano6060116
Makadia, H. K., & Siegel, S. J. (2011). Poly Lactic-co-Glycolic Acid (PLGA) as Biodegradable Controlled Drug Delivery Carrier. Polymers (Basel), 3(3), 1377-1397. doi:10.3390/polym3031377
Mancini, M. C., Kairdolf, B. A., Smith, A. M., & Nie, S. (2008). Oxidative Quenching and Degradation of Polymer-Encapsulated Quantum Dots: New Insights into the Long-Term Fate and Toxicity of Nanocrystals in Vivo. Journal of the American Chemical Society, 130(33), 10836-10837. doi:10.1021/ja8040477
Nehilla, B. J., Allen, P. G., & Desai, T. A. (2008). Surfactant-Free, Drug-Quantum-Dot Coloaded Poly(lactide-co-glycolide) Nanoparticles: Towards Multifunctional Nanoparticles. ACS Nano, 2(3), 538-544. doi:10.1021/nn700281b
Raoufinia, R., Mota, A., Keyhanvar, N., Safari, F., Shamekhi, S., & Abdolalizadeh, J. (2016). Overview of Albumin and Its Purification Methods. Adv Pharm Bull, 6(4), 495-507. doi:10.15171/apb.2016.063
Tang, Q., Qian, Y., Yang, D., Qiu, X., Qin, Y., & Zhou, M. (2020). Lignin-Based Nanoparticles: A Review on Their Preparations and Applications. Polymers (Basel), 12(11). doi:10.3390/polym12112471 Wada, Y., Takeda, Y., & Kuwahata, M. (2017). Potential Role of Amino Acid/Protein Nutrition and Exercise in Serum Albumin Redox State. Nutrients, 10(1). doi:10.3390/nu10010017
Weaver, J., Zakeri, R., Aouadi, S., & Kohli, P. (2009). Synthesis and characterization of quantum dot—polymer composites. Journal of materials chemistry, 19, 3198-3206. doi:10.1039/b820204d
Williams, A. T. R., Winfield, S. A., & Miller, J. N. (1983). Relative fluorescence quantum yields using a computer-controlled luminescence spectrometer. Analyst, 108(1290), 1067-1071. doi:10.1039/AN9830801067
Yan, R., Yu, B. Q., Yin, M. M., Zhou, Z. Q., Xiang, X., Han, X. L., . . . Jiang, F. L. (2018). The interactions of CdTe quantum dots with serum albumin and subsequent cytotoxicity: the influence of homologous ligands. Toxicol Res (Camb), 7(2), 147-155. doi:10.1039/c7tx00301c
Yin, W., Liu, H., Yates, M. Z., Du, H., Jiang, F., Guo, L., & Krauss, T. D. (2007). Fluorescent Quantum Dot-Polymer Nanocomposite Particles by Emulsification/Solvent Evaporation. Chemistry of Materials, 19(12), 2930-2936. doi:10.1021/cm070214g

### INCORPORATIONS BY REFERENCE

The complete disclosures of all references cited in this specification are hereby incorporated by reference. Also incorporated by reference is the complete disclosure of U.S. the priority provisional application Ser. No. 63/060,214. In the event of an otherwise irreconcilable conflict, the present specification shall take precedence over material incorporated by reference.

What is claimed:

1. A protected quantum dot composition, comprising quantum dots and a graft copolymer; wherein:
(a) said quantum dots have a mean diameter from 2 nm to 20 nm; said quantum dots comprise a semiconductor; said quantum dots luminesce with excitation in the ultraviolet spectrum and emission in the ultraviolet, visible, or infrared spectrum; and said quantum dots are nontoxic to mammals;
(b) said graft copolymer comprises a hydrophobic polymer domain and a hydrophilic polymer or protein domain, wherein said hydrophobic polymer domain and said hydrophilic polymer or protein domain are covalently bonded to one another; and wherein each of said hydrophobic polymer and said hydrophilic polymer or protein is nontoxic to mammals;
(c) said graft copolymer comprises core-shell nanoparticles; wherein the inner core of said core-shell nanoparticles predominantly comprises said hydrophobic polymer domain, and wherein the outer shell of said core-shell nanoparticles predominantly comprises said hydrophilic polymer or protein domain; whereby said hydrophobic polymer domain in said outer shell makes said composition overall hydrophilic;
(d) said nanoparticles have a mean diameter from 70 nm to 500 nm;
(e) said quantum dots are predominantly located inside said nanoparticles; wherein said quantum dots associate primarily with the inner, hydrophobic core of said nanoparticles if the surface of said quantum dots is hydrophobic and is not electrostatically charged; and wherein said quantum dots associate primarily with the outer, hydrophilic shell of said nanoparticles if the surface of said quantum dots is hydrophilic or is electrostatically charged; and
(f) said composition has the property that, when said composition is in an aqueous environment, then as compared to free quantum dots that are otherwise chemically identical but that lack the graft copolymer and the nanoparticles, the degradation rate of said quantum dots within said composition is slower by a factor of at least 1.25.

2. The composition of claim 1, wherein said hydrophobic polymer domain comprises one or more polymers selected from the group consisting of poly(lactic-co-glycolic) acid (PLGA), polystyrene, polyhydroxyalkanoates, polylactic acid, poly glycolic acid, poly(methyl methacrylate), ammonio methacrylate, polystyrene, poly(styrene-co-maleic anhydride), polyethylene, and poly(propylene oxide).

3. The composition of claim 1, wherein said hydrophilic polymer or protein domain comprises one or more polymers or proteins selected from the group consisting of zein, soy protein, poly(ethylene glycol) (PEG), poly(vinyl alcohol) (PVA), poly(glutamic acid), sodium lignosulfonate (SLGN), bovine serum albumin (ALB), alkaline lignin, polyacrylamide, polyethyleneimine, collagen, substituted or unsubstituted cellulose, substituted or unsubstituted starch, and polynucleotides.

4. The composition of claim 1, wherein said quantum dots comprise one or more semiconductors selected from the group consisting of ZnSe, ZnSe:Mn, ZnSe:Cu, ZnSe:Ag, other doped ZnSe semiconductors, graphene QDs, carbon QDs, far infrared QDs, other zinc-based QDs, InP, CuInSe₂, AgInSe₂, CuInS₂, AgInS₂, other metal-based QDs, and other III-V semiconductors.

5. The composition of claim 1, wherein said nanoparticles have a mean diameter from 100 nm to 250 nm.

6. The composition of claim 1, wherein the surface of said quantum dots is hydrophobic, and wherein said quantum dots associate primarily with the inner core of said core-shell nanoparticles.

7. The composition of claim 1, wherein the surface of some of said quantum dots is hydrophobic, wherein the surface of some of said quantum dots is hydrophilic, wherein said quantum dots with hydrophobic surfaces associate primarily with the inner core of said core-shell nanoparticles, and wherein said quantum dots with hydrophilic surfaces associate primarily with the outer shell of said core-shell nanoparticles.

8. The composition of claim 1 wherein, when said composition is in an aqueous environment, then as compared to free quantum dots that are otherwise chemically identical but that lack the graft copolymer and the nanoparticles, the degradation rate of said quantum dots within said composition is slower by a factor of at least 2.

9. The composition of claim 1 wherein, when said composition is in an aqueous environment, then as compared to free quantum dots that are otherwise chemically identical but that lack the graft copolymer and the nanoparticles, the degradation rate of said quantum dots within said composition is slower by a factor of at least 5.

10. The composition of claim 1, wherein said hydrophobic polymer domain comprises poly(lactic-co-glycolic) acid (PLGA); wherein said hydrophilic polymer domain comprises alkaline lignin or sulfonated lignin; wherein said quantum dots comprise a doped ZnSe semiconductor; wherein said nanoparticles have a mean diameter from 100 nm to 250 nm; wherein the surface of said quantum dots is hydrophobic; wherein said quantum dots associate primarily with the inner core of said core-shell nanoparticles; and wherein, when said composition is in an aqueous environment, then as compared to free quantum dots that are otherwise chemically identical but that lack the graft copolymer and the nanoparticles, the degradation rate of said quantum dots within said composition is slower by a factor of at least 2.

11. The composition of claim 1, wherein said composition is a solid-state composition.

12. An aqueous mixture comprising an aqueous suspension of the composition of claim 1.

13. The composition of claim 1, wherein said hydrophilic polymer domain, said hydrophobic polymer domain, or both comprises a biopolymer.

14. The composition of claim 1, wherein said polymer domains are not crosslinked.

15. The composition of claim 1, wherein at least one of said polymer domains is crosslinked.

16. The composition of claim 1, wherein over 50% of said nanoparticles each contain a plurality of said quantum dots.

17. The composition of claim 1, wherein the quantum yield of said quantum dots within said composition is 60% or greater of the quantum yield of free quantum dots that are otherwise chemically identical but that lack the graft copolymer and the nanoparticles.

18. The composition of claim 1, wherein the quantum yield of said quantum dots within said composition is 75% or greater of the quantum yield of free quantum dots that are otherwise chemically identical but that lack the graft copolymer and the nanoparticles.

19. The composition of claim 1, wherein the quantum yield of said quantum dots within said composition is 90% or greater of the quantum yield of free quantum dots that are otherwise chemically identical but that lack the graft copolymer and the nanoparticles.
